(19) United States
(12) Patent Application Publication
(10) Pub. No.: US 2007/0073050 A1
(43) Pub. Date: Mar. 29, 2007

(54) Process for the preparation of doxifluridine

(76) Inventors: Giorgio Bertolini, Milano (IT); Marco Frigerio, Milano (IT)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/576,598

(22) PCT Filed: Oct. 21, 2004
(86) PCT No.: PCT/IB04/03448

§ 371 Date: Apr. 21, 2006

(30) Foreign Application Priority Data
Oct. 22, 2003 (IT) MI2003A002059

(51) Int. Cl. C07H 19/12

(52) U.S. Cl. 536/28.3; 536/55.3

(57) ABSTRACT

A process is described for the preparation of doxifluridine of formula 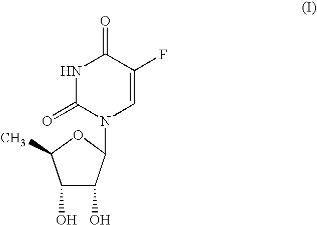 at high yields and with reduced formation of impurities, which comprises the reaction of coupling of a compound of formula (III) with a compound of formula (IV), as defined in the description, in the presence of a Lewis acid and in an inert organic solvent, characterized in that said Lewis acid is added at a temperature below 0° C.

The present invention relates to a process for the preparation of doxifluridine and more particularly to a process of preparation characterized by high yields and reduced formation of impurities.

### STATE OF THE ART

Doxifluridine, or 5′-deoxy-5-fluorouridine, is a known compound with anticytostatic activity, currently used as an antineoplastic agent (Merck Index No. 3471, 13^{th} Ed. 2001), of formula I:

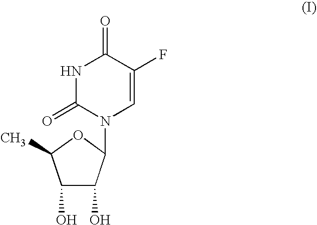

Various processes for the production of doxifluridine are known, and that described for example in U.S. Pat. No. US4,340,729 is of particular importance in the present context.

Thus, this patent describes a process for production of doxifluridine that comprises essentially the coupling reaction between a ribose derivative modified at 5′ and suitably protected (III), and activated 5-fluorouracil (IV), according to the scheme:

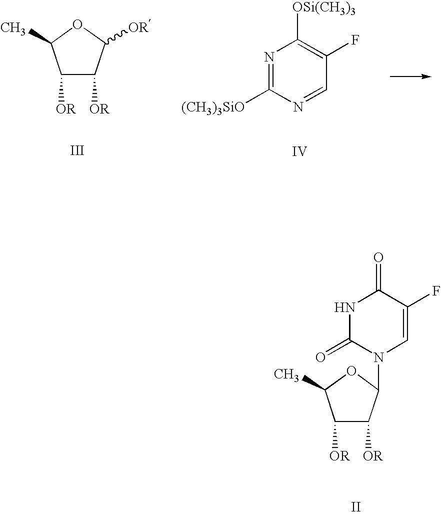

Said coupling reaction (column 3, lines 27-36) takes place in the presence of a Lewis acid, such as trimethylsilyltrifluoromethanesulphonate or fin tetrachloride, in an inert organic solvent, at or below room temperature, preferably cooling in ice.

The experimental part describes (column 5, lines 34-60), in particular, the coupling reaction between 5-deoxy-1,2,3-tri-O-acetyl-D-ribofuranoside (III, R═R′═acetyl) and 2,4bis(trimethylsilyl)-5-fluorouracil, catalysed by trimethylsilyltrifluoromethane-sulphonate, carried out at the temperature of an ice bath.

However, the reaction as described in the US patent is not entirely satisfactory, especially with a view to its large-scale use.

In fact, both on repeating the same reaction in the presence of trimethylsilyltrifluoro-methanesulphonate and on using, alternatively, tin tetrachloride as catalyst, we observe the formation of an impurity in significant amounts, an impurity that leads, first of all, to a reduction of the yields and, moreover, complicates the procedures for isolating and purifying the final product. This side reaction, which already occurs in the conditions described in U.S. Pat. No. 4,340,729, is especially pronounced if the reaction is catalysed with tin tetrachloride: in this case, in fact, a very complex reaction mixture is obtained, where the by-product, which is difficult to remove, represents approx. 11% of said mixture, whereas the desired product (II, R═acetyl) comes to at most 70%, calculated by area (HPLC) (see Table 1).

We found, surprisingly, that it is possible to increase the yields of this coupling reaction significantly and reduce the formation of by-products, in a simple way that can be applied industrially, thus making it possible to isolate the raw product without excessive manipulations and at a purity such that it can be used directly for the subsequent stage of deprotecton. It is obvious to a person skilled in the art that this simplification leads to a considerable reduction both of process times and costs when applied on an industrial scale.

### DESCRIPTION OF THE INVENTION

Therefore the object of the present invention is a process for the preparation of doxifluridine of formula 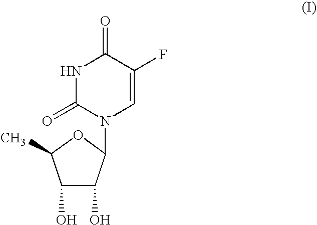
which comprises the reaction of coupling of a compound of formula

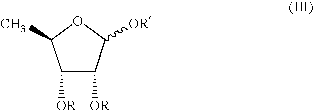

in which

R represents a linear or branched aliphatic C₁-C₅ acyl or benzoyl, optionally substituted with C₁-C₅ alkyls, C₁-C₅ alkoxyls or halogens,

R′ represents R or a linear or branched C₁-C₅ alkyl, R and R′ being identical or different,

with a compound of formula

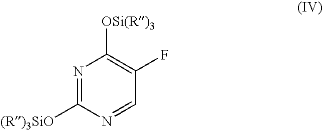

in which

R″ identical or different, represent a C₁-C₆ alkyl or a phenyl,

in the presence of a Lewis acid and in an inert organic solvent to give the compound of formula

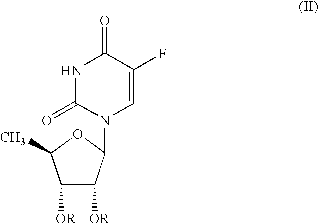

in which R has the meanings stated above,

characterized in that said Lewis acid is added at a temperature below 0° C., preferably below −10° C., more preferably between about −15 and −20° C.

Preferably, when addition of the catalyst is completed, the reaction mixture is then maintained at the same temperature for a variable time, preferably for at least about 2 h, more preferably at least about 4 h.

The starting compounds of formula III, in which R and R′ preferably represent acyl, more preferably acetyl, and IV, in which the R″ are preferably identical to one another and preferably represent methyl, can be prepared according to known methods, for example as described in U.S. Pat. No. 4,340,729.

The Lewis acid used is preferably trimethylsilyltrifluoromethanesulphonate or tin tetrachloride, more preferably tin tetrachloride.

Inert organic solvents that are preferred according to the present invention are chlorinated solvents, preferably methylene chloride, or aromatic solvents, preferably toluene, more preferably the chlorinated solvents.

The coupling product II of the present reaction, in which R preferably represents acyl, more preferably acetyl, can then be submitted directly to the appropriate known reactions of deprotection for the removal of the specific, preselected protecting groups, for example as described in U.S. Pat. No. 4,340,729, to give doxifluridine.

According to a preferred embodiment of the present invention, tin tetrachloride is added to a mixture of the compound of formula IV, in which R″═methyl, and of the compound of formula III, in which R═R′═acetyl, in a chlorinated solvent, cooled to a temperature below −10° C., and the mixture is held at said temperature, while stirring, for at least 2 h, optionally then leaving the mixture to react over night at room temperature.

The following examples are now supplied for better illustration of the present invention:

### EXPERIMENTAL SECTION

### EXAMPLE 1

### Preparation of 2′,3′-diacetyl-5′-deoxy-5-fluorouridine (II, R═acetyl)

A) Preparation according to the invention Suspend 5-fluorouracil (65 g), trimethylchlorosilane (48 ml) and hexamethyl-disilazane (76 ml) in methylene chloride (520 ml) and heat the reaction mixture under reflux for 4 h. Cool the suspension thus obtained to 20-25° C. and add 5-deoxy-1′,2′,3′-triacetyl-D-ribose (130 g). Cool the reaction mixture to −20/−15° C. and add the tin tetrachloride (58 ml) slowly (in approx. 2 h), maintaining the temperature between −20° C. and −15° C. Stir the reaction mixture between −20° C. and −15° C. for at least 4 h, then leave the temperature to rise slowly from −20/−15° C. to 20° C. over night, with stirring.
Then cool the mixture to 0-5° C. and slowly add, dropwise, at this temperature, a solution of 36% concentrated hydrochloric acid (200 ml) in water (1300 ml). Separate the two phases and extract the aqueous phase twice with methylene chloride (2×250 ml). Combine the organic phases and treat with water (1000 ml) and add sodium bicarbonate (approx. 12 g) to pH 7. Separate the two phases and dry the organic phase over magnesium sulphate (5 g). After filtration of the magnesium sulphate, concentrate the organic phase at reduced pressure and submit the residue thus obtained directly to the next stage of deprotection.
B) Preparation according to the conditions described in U.S. Pat. No. 4,340,729 The reaction was repeated on the same quantities and with the same reactants described above, but changing the temperature of addition of the tin tetrachloride to the mixture from −20/−15° C. to 0/+15° C. (ice bath).

The results of the two tests, analysed by HPLC (column: Zorbax SB-AQ 100−4.6 −3.5 μm; mobile phase A: ammonium acetate buffer in water 6.0 g/l with pH corrected to 5.6 with acetic acid; mobile phase B: acetonitrile-methanol-water 45:45:10; flow rate 0.8 ml/min; detector 280 nm; gradient time zero 98% phase A, time 2 minutes 98% phase A, time 20 minutes 35% phase A) are shown in the following Table 1:

TABLE 1

| | | Composition of the reaction mixture | | | |
|---|---|---|---|---|---|
| | | (area % HPLC) | | | |
| | | 5-fluoro- | | Main | Ratio |
| Example | Temperature | uracil | II | impurity | II:impurity |
| 1A | −20/−15° C. | 8% | 90% | 1% | 90:1 |
| 1B | 0/15° C. | 11% | 70% | 11% | 6.4:1 |

As can be seen, the reaction carried out according to the present invention (1A) shows a surprising decrease of the main impurity (retention time 23.8 minutes) from 11% to 1% and, at the same time, a significant increase in the desired product II, from 70% to 90%, relative to the reaction carried out in the conditions described in the prior art (1B).
C) Preparation of doxifluridine (I) Dissolve the raw residue of 2′,3′-diacetyl-5′-deoxy-5-fluorouridine (II, R═acetyl), obtained as described above, in methanol (100 ml) and evaporate the solvent at reduced pressure. Then dissolve the residue in methanol (1500 ml) and add a 25% solution of sodium methoxide in methanol (98 g). Stir the reaction mixture at 20-25° C. for 3 h, then add 36% concentrated hydrochloric acid (approx. 50 ml) without exceeding 10° C. to pH 4.0/4.2. Evaporate the solvent at reduced pressure and dissolve the residue in isopropanol (2×150 ml), then evaporate the solvent at reduced pressure again. Repeat this operation twice, then dissolve the residue in isopropanol (3600 ml) and heat the suspension under reflux. Filter the undissolved salts while hot, and concentrate the solution at 50° C. and at reduced pressure to approx. 2400 ml. Cool the suspension thus obtained to 0/5° C. and stir it at this temperature for one hour. Filter the solid, wash it with cold isopropanol (200 ml) obtaining, after drying at 50° C., 91 g of pure doxifluridine (molar yield in the two passes 74%).

### EXAMPLE 2

### Preparation of 2′,3′-diacetyl-5′-deoxy-5-fluorouridine (II, R═acetyl)

Following the procedure described in Example 1, but using trimethylsilyltrifluoro-methanesulphonate as catalyst, the coupling reactions according to the invention (−20/−15° C.) (2A) and according to U.S. Pat. No. 4,340,729 (0/+15° C.) (2B) were repeated. Similarly to Example 1, the reactions were monitored at successive intervals by HPLC, in the same conditions. After two hours of reaction, the ratios between the HPLC areas of the desired product (II, R═acetyl) relative to the impurity (r.t.═23.8 minutes) had the following values:

TABLE 2

| | | Ratio 2′,3′-diacetyl-5′-deoxy- |
|---|---|---|
| Test | Temperature | 5-fluorouridine:impurity |
| 2A | −20/−15° C. | 10:1 |
| 2B | 0/15° C. | 3.4:1 |

The surprising improvement obtained with the present invention, in terms of reduction of the formation of impurity, relative to that described in the prior art, is also confirmed in this case.

1. A process for preparing a compound of formula 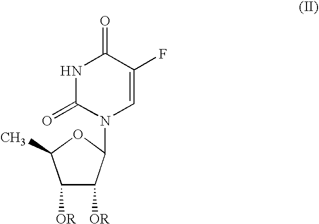
in which R represents linear or branched C₁-C₅ aliphatic acyl or benzoyl, optionally substituted with C₁-C₅ alkyls, C₁-C₅ alkoxyls or halogens, which comprises the reaction of coupling of a compound of formula 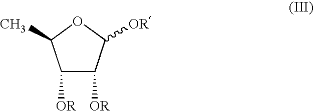
in which
R represents a linear or branched C₁-C₅ aliphatic acyl or benzoyl, optionally substituted with C₁-C₅ alkyls, C₁-C₅ alkoxyls or halogens,
R′ represents R or a linear or branched C₁-C₅ alkyl, with a compound of formula 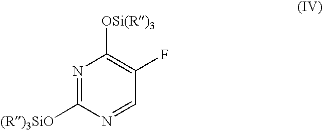
in which R″, being identical or different, represents a C₁-C₆ alkyl or a phenyl, in the presence of a Lewis acid catalyst and in an inert organic solvent, wherein said Lewis acid catalyst is added to a reaction mixture of the compounds of formula (III) and formula (IV) at an addition temperature below −10° C.

2. The process according to claim 1 in which said addition of the Lewis acid catalyst is carried out at an addition temperature between approx. −15 and −20° C.

3. The process according to claim 1 in which, on completion of said addition of said Lewis acid catalyst, the reaction mixture is held further at the addition temperature.

4. The process according to claim 1 in which R and R′ represent acyl, and R″ represents methyl.

5. The process according to claim 1 in which said Lewis acid is selected from the group consisting of trimethylsilyltrifluoromethanesulphonate, tin tetrachloride, and mixtures thereof.

6. The process according to claim 1 in which said inert organic solvent is selected from the group consisting of a chlorinated solvent aromatic solvent, and mixtures thereof preferably.

7. The process according to claim 1 in which said compound of formula II, 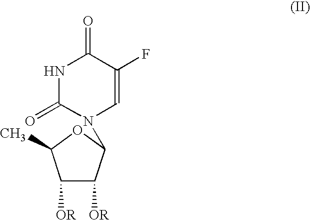
in which R represents a linear or branched C₁-C₅ aliphatic acyl or benzoyl, optionally substituted with C₁-C₅ alkyls, C₁-C₅ alkoxyls or halogens, and the compound of formula (II) is further submitted to a reaction of deprotection to give doxifluridine of formula I

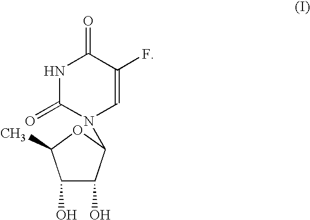

8. A process for the preparation of doxifluridine of formula (I) 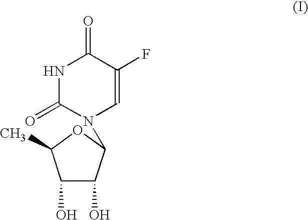
said process comprising coupling a compound of formula (III) 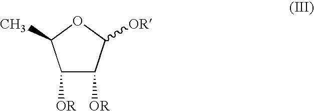
where R represents a linear or branched C₁-C₅ aliphatic acyl or benzoyl, optionally substituted with C₁-C₅ alkyls, C₁-C₅ alkoxyls or halogens, with a compound of formula (IV) 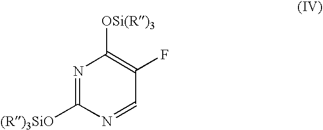
where R″ being identical or different represents a C₁-C₆ alkyl or a phenyl, said coupling reaction taking place in the presence of a Lewis acid being added at a temperature of less than −10° C. to provide a compound of formula (II) 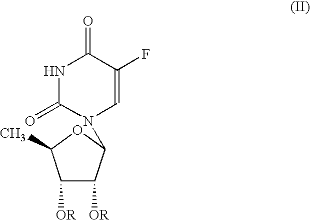
and deprotecting the compound of formula (II) to provide the compound of formula I

9. The process of claim 1, wherein R and R′ are acetyl and R″ is methyl.

10. The process of claim 1, wherein the Lewis acid is tetrachloride.

11. The process of claim 1, wherein the inert organic solvent is a chlorinated solvent.
